(19) United States
(12) Patent Application Publication
(10) Pub. No.: US 2011/0039346 A1
(43) Pub. Date: Feb. 17, 2011

(54) DRUG DETECTION STRAW

(76) Inventors: Michael BRADLEY, Dallas, TX (US); Anthony LEWIS, Dallas, TX (US)

Correspondence Address: LAW OFFICE OF JERRY D. HAYNES , P.A, P.O. Box 350392, Fort Lauderdale, FL, 33335, US

(21) Appl. No.: 12/541,152

(22) Filed: Aug. 13, 2009

(51) Int. Cl. G01N 21/78; G01N 31/22; G01N 33/15

(52) U.S. Cl. 436/98; 436/164; 436/127; 422/425

(57) ABSTRACT

The present invention relates to a drug detection device for detecting the presence of a date rape drug in a beverage comprising: a straw; and a chemical reagent, where said chemical reagent coats the surface of the straw and changes color upon contact with a date rape drug. In one exemplary embodiment, the drug detection device may be used to test for Gamma Hydoxybutyrate (GHB) or ROHYPNOL® (Flunitrazepam). A stirring straw may be used as a component of the drug detection device as opposing to a conventional straw. The present invention also includes a method of date rape drug detection comprising the steps of: coating a stirring device with a chemical reagent; inserting the stirring device in a beverage; and observing the stirring device to determine an indication of the presence of a date rape drug.

### BACKGROUND OF THE INVENTION

1. Field of Invention

The present invention relates to a straw or stirring device that is utilized to detect the presence of drugs in a liquid drink.

2. Description of Related Art

Certain drugs are available to the public for purposes contrary to their intended use. Many of these drugs are categorized as date rape drugs where the drugs are added to an alcoholic beverage and upon consumption induce a sedated type of consciousness. Such date rape type drugs include Gamma Hydoxybutyrate, GHB, along with a drug by the trade name of Rohypnol® also known as Roofies. Many of these drugs are approved medications for use to help with sleeping disorders. However these drugs are being used for illicit purposes in order to spike an unsuspected female's drink in order to place the woman in a disoriented and unconscious position for unsavory purposes.

Many times women or young girls are subjected to these drugs without their knowledge and therefore are accosted and raped without the knowledge of the incident ever occurring. Mixture of these drugs with alcohol can cause a woman to pass out for eight to 24 hours without knowledge of activities taking place during the period of unconsciousness. Consequently these drugs have been characterized as date rape drugs and their reported use has escalated over recent years.

It would be therefore advantageous for a woman to have a device or means to test a alcoholic beverage that may have been out of her possession and control for any date rape type of drug that may be been spiked into the drink. One particular means of testing has been developed and disclosed in U.S. Pat. No. 7,238,533 which discloses the placement of a chemically reactive substance on the layer of at least one fingernail of the user where the substance may be blended with fingernail polish. The user of the substance disclosed in the '533 patent simply places their fingernail within the drink and if the drink causes the nail polish to change a particular color there is indication of presence of a suspected drug in the beverage.

### SUMMARY OF THE INVENTION

The present invention relates to a drug detection device for detecting the presence of a date rape drug in a beverage comprising: a straw; and a chemical reagent, where said chemical reagent coats the surface of the straw and changes color upon contact with a date rape drug. In one exemplary embodiment, the drug detection device may be used to test for Gamma Hydoxybutyrate (GHB) or Rohypnol®. A stirring straw may be used as a component of the drug detection device as opposing to a conventional straw. The present invention also includes a method of date rape drug detection comprising the steps of: coating a stirring device with a chemical reagent; inserting the stirring device in a beverage; and observing the stirring device to determine an indication of the presence of a date rape drug.

### BRIEF DESCRIPTION OF DRAWINGS

FIG. 1 depicts the chemical detecting stirring device according to the present invention.

### DETAILED DESCRIPTION

The present invention discloses a stirring straw or drinking straw that may be utilized to test and detect the presence of date rape drugs in a liquid beverage. According to present invention, a straw or a stir straw may be coated with a reagent that chemically reacts with date rape drugs in order to detect the presence thereof. The reagent on the straw or stirring straw reacts with the date rape drug and changes the color of the straw or stirring straw in order to alert the user of the presence of a date rape drug. The testing straw according to the present invention enables the user to detect the presence of a date rape drugs and therefore avoid the consumption thereof while enjoying an alcoholic beverage in a public bar, nightclub or other establishment.

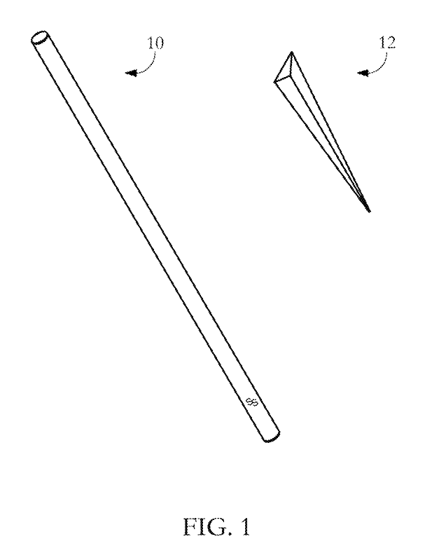

The FIG. 1 depicts a Straw 10 and alternatively a Stirring Straw 12 that may be coated with a chemically reactive substance that detects the presence of date rape drugs. The specific chemically reactive substance are well known in the art and therefore not listed in detail as part of this application. When using the straw 10 or stirring straw 12 according to the present invention, the user may simply insert or dip the straw into the drink without being noticed and then retract the straw to detect if a color change occurs before consuming the drink that has been subjected to the testing. The blending of this chemically reactive substance with the drink therefore provides a means to detect the insertion of date rape drugs in a beverage.

Using such a device would be helpful and advantageous to any woman that may be at a bar or nightclub either unaccompanied or for some reason leaves their drinks unattended for any period of time. The drug detecting Straw 10 or Stirring Straw 12 may be easily stored in the user's purse or handbag and used when necessary when a drink is left unattended. The use of this device may help prevent many incidents of date rape and discourage the use of drugs in this manner. Also the use of this device helps a victim of a possible date rape drug to alert authorities of individuals who might use these types of drugs for date rape purposes.

The instant invention has been shown and described in what is considered to be the most practical and preferred embodiments. It is recognized, however, that departures may be made there from within the scope of the invention and that obvious modifications will occur to a person skilled in the art.

1. A drug detection device for detecting the presence of a date rape drug in a beverage comprising:
a. a drinking straw; and
b. a chemical reagent, where said chemical reagent coats the surface of the straw and changes color upon contact with a date rape drug.

2. The drug detection device according to claim 1, where said date rape drug is at least one of Gamma Hydoxybutyrate (GHB) and ROHYPNOL® (Flunitrazepam).

3. A drug detection device for detecting the presence of date rape drug in a beverage comprising:
a. a stirring straw; and
b. a chemical reagent, where said chemical reagent coats the surface of the straw and changes color upon contact with a date rape drug.

4. The drug detection device according to claim 3, where said date rape drug is at least one of Gamma Hydoxybutyrate (GHB) and ROHYPNOL® (Flunitrazepam).

5. A method of date rape drug detection comprising the steps of:
a. coating a stirring device with a chemical reagent;
b. inserting the stirring device in a beverage; and
c. observing the stirring device to determine an indication of the presence of a date rape drug.

6. The method according to claim 5, where the date rape drug is at least one of Gamma Hydoxybutyrate (GHB) and ROHYPNOL® (Flunitrazepam).

7. The method according to claim 5, where the stirring device includes at least a straw and a stirring straw.
